(19) United States
(12) Patent Application Publication
(10) Pub. No.: US 2010/0042018 A1
(43) Pub. Date: Feb. 18, 2010

(54) DEVICE FOR TREATING CELLULITE AND FATTY MASSES

(76) Inventor: Alain Kleinsinger, Neuilly-Sur-Seine (FR)

Correspondence Address: Blakely, Sokoloff, Taylor & Zafman LLP, 1279 Oakmead Parkway, Sunnyvale, CA, 94085-4040, US

(21) Appl. No.: 12/526,939

(22) Filed: Aug. 12, 2009

Related U.S. Application Data
(63) Continuation-in-part of application No. PCT/EP2008/051677, filed on Feb. 12, 2008.

(30) Foreign Application Priority Data
Feb. 12, 2007 (FR) 0753195

(51) Int. Cl. A61N 1/36; A61H 1/00

(52) U.S. Cl. 601/2; 607/3

(57) ABSTRACT

A device (10) for treating cellulite and fatty masses comprising means (200) for forming a volume of adipose tissue on a zone to be treated of the body of a subject, means (300) for generating ultrasound and means (400) for generating electric pulses on the volume of adipose tissue formed or at the periphery of the volume of adipose tissue formed, said device (10) being characterised in that the means for generating ultrasound (300) are supported by a supple membrane (500) connected to the means (200) for forming a volume of adipose tissue, and adapted so that, when the device (10) is placed on the zone to be treated of the body of a subject, the means (300) for generating ultrasound remain in contact with the volume of adipose tissue to be treated, and in that the means for generating electric pulses (400) are placed on or at the periphery of a face of the device (10) in contact with the zone to be treated.

The invention relates to processes and devices for treating cellulite and fatty masses.

Cellulite is defined as hydro-lipodystrophia, that is, water infiltration, due to the dilation of blood capillaries, and accumulation of fats, accompanied by morphological modification of fatty cells (adipocytes) causing thickening and disruption of fibrous tissue.

Oedema forms, increasing water pressure and compressing fibres and cells as well as blood vessels. Venous and lymphatic circulation is affected, causing a state of “asphyxia” of the connecting tissue and its premature ageing, accompanied by fibrosis with cutaneous thickening, responsible for the characteristic appearance of “orange peel”.

The cellulite is localised in preplaced regions of the body of a subject, especially at the buttocks, hips and thighs.

Conventionally, the usual method for treating cellulite is liposuction. Canulas are used which penetrate the body of the subject to suction out subcutaneous fatty cells in the abovementioned regions of the body of the subject.

However, this method is invasive, destructive, painful and has risks including the death of the subject.

Other alternatives to liposuction exist to resolve this aesthetic problem and treat the accentuation of adipose tissues, for example by activating local venous circulation, by dissolving the fat in the adipocytes or even by disrupting the pocket of fatty cells.

Using creams to dissolve fatty cells or even capsules to ingest to improve lymphatic drainage of lipidic waste can be mentioned, but these treatments are barely efficacious.

The use of ultrasound devices is also known. The generation of ultrasound in the body of the subject transmits acoustic vibrations to the fatty cells, activating lipidic exocytosis of adipocytes, that is, rejection of fats from fatty cells.

However, even though ultrasound has a heightened affinity for adipose tissue, their intensity must be limited so as not to provoke heating of the tissues, particularly on the skin. The majority of current ultrasound devices generates reduced energy to transmit to the tissues, thus limiting the efficacy of treatment.

The expelled fats are then drained out by the lymphatic and venous system of the subject and eliminated naturally.

However, this draining is often insufficient and part of the lipids stagnates in the interstitial spaces of the fatty cells and are progressively reabsorbed by the latter.

These alternatives to liposuction have only temporary action on cellulite, and are unsatisfactory.

An aim of the present invention is to provide an alternative which is efficacious, reliable, economical, non invasive and devoid of risk, to the liposuction method in treating cellulite and fatty masses.

Another aim of the present invention is to propose a device for treating cellulite and fatty masses which increases and accelerates lipidic exocytosis of fatty cells.

Another aim of the present invention is to propose a device for treating cellulite and fatty masses which boosts energy transmitted to adipose tissues while remaining within regulatory limits of intensity of ultrasound emissions.

Another aim of the present invention is to propose a device for treating cellulite and fatty masses which limit any local or even temporary accumulation of lipids expelled from fatty cells.

Another aim of the present invention is to propose a device for treating cellulite and fatty masses which improves the persistence of results of treatment over time.

Another aim of the present invention is to propose a device for treating cellulite and fatty masses which can treat subjects, irrespective of the elasticity of their skin.

Another aim of the present invention is to propose a device for treating cellulite and fatty masses which proposes painless treatment.

These aims are attained according to the invention due to a device for treating cellulite and fatty masses comprising means for forming a volume of adipose tissue on a zone to be treated of the body of a subject, means for generating ultrasound and means for generating electric pulses on the volume of adipose tissue formed, in which the means for generating ultrasound are supported by a supple membrane connected to the means for forming a volume of adipose tissue, and adapted so that, when the device is placed on the zone to be treated of the body of a subject, the means for generating ultrasound remain in contact with the volume of adipose tissue to be treated, and in that the means for generating electric pulses are placed opposite the device in contact with the zone to be treated.

The invention also relates to a device for treating cellulite and fatty masses comprising means for forming a volume of adipose tissue on a zone to be treated of the body of a subject, means for generating ultrasound and means for generating electric pulses on the volume of adipose tissue formed, in which the means for generating ultrasound and the means for generating electric pulses are supported by a supple membrane connected to the means for forming a volume of adipose tissue, and adapted so that when the device is placed on the zone to be treated of the body of a subject the means for generating ultrasound and the means for generating electric pulses remain in contact with the volume of adipose tissue to be treated.

The invention further relates to a process for treating cellulite and fatty masses, characterised in that it comprises a step of forming a volume of adipose tissue on a zone to be treated of the body of a subject followed by a step of placing a supple membrane connected to a support in contact with the volume of adipose tissue thus formed, said supple membrane supporting means for generating ultrasound, and said supple membrane or said support supporting means for generating electric pulses and followed by a step for generating ultrasound and for generating electric pulses on the volume of adipose tissue formed.

Advantageously, the means for generating ultrasound and the means for generating electric pulses are supported by a supple membrane designed to be placed in contact with the volume of adipose tissue formed.

Other characteristics, aims and advantages of the invention will emerge from the following detailed description, which is purely illustrative and non-limiting and must be read in conjunction with the attached diagrams, in which:

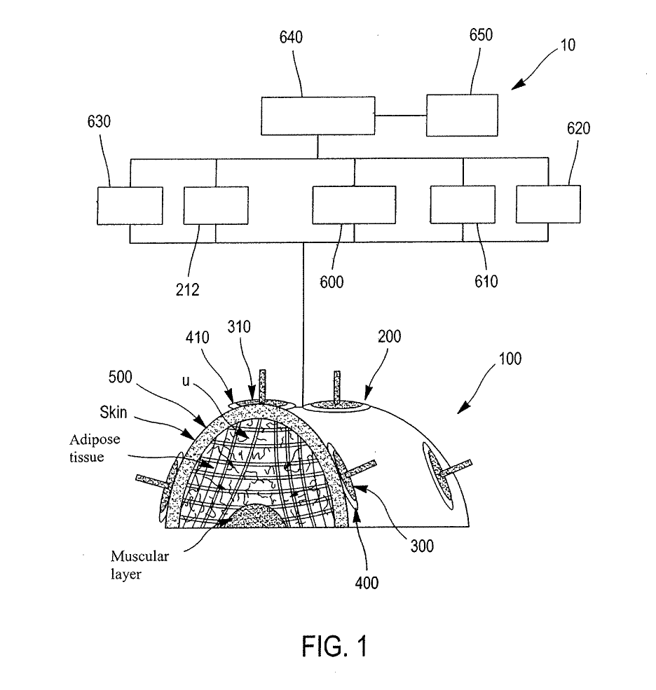

FIG. 1 illustrates a schematic view of a device for treating cellulite and fatty masses according to the invention;

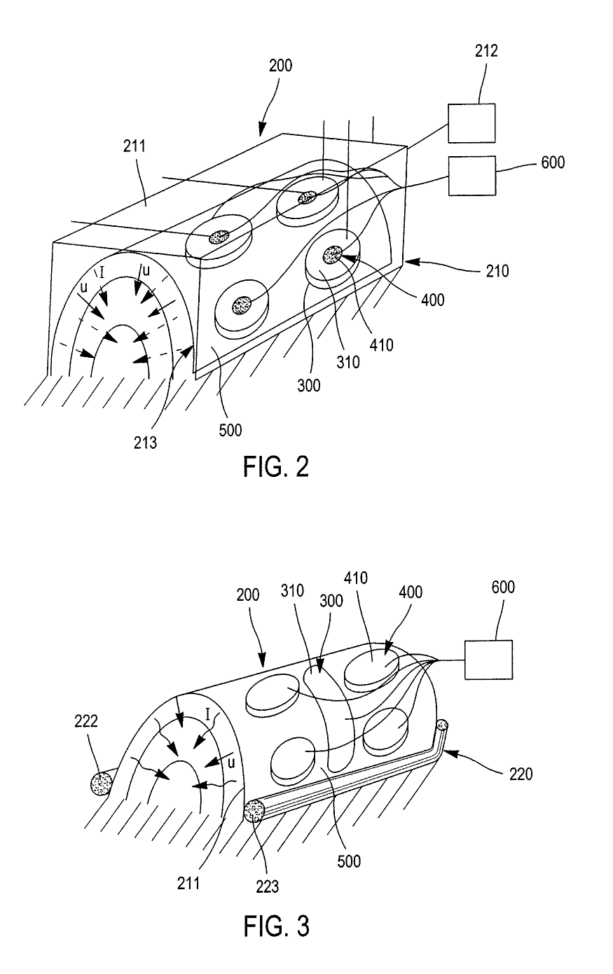

FIG. 2 illustrates an embodiment of means for three-dimensionally implementing the device of FIG. 1;

FIG. 3 illustrates another embodiment of means for three-dimensionally implementing the device of FIG. 1;

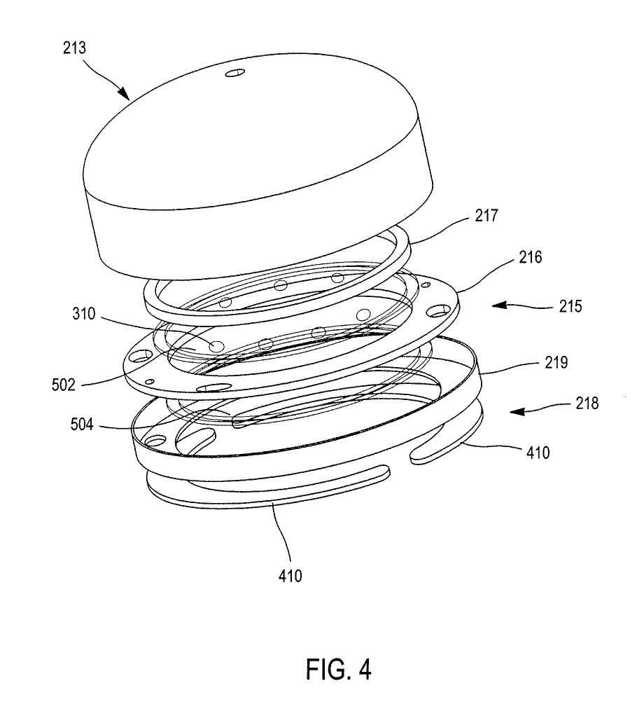

FIG. 4 illustrates an exploded view of another embodiment of means for three-dimensionally implementing the device of FIG. 1;

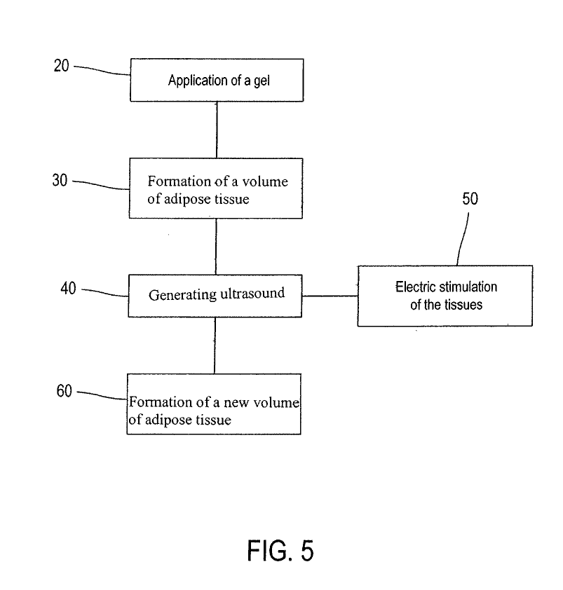

FIG. 5 illustrates a display diagram of a process for treating cellulite and fatty masses implemented by the device of FIG. 1.

### 1. STRUCTURE OF A DEVICE FOR TREATING CELLULITE AND FATTY MASSES

A device 10 designed to treat cellulite and fatty masses is illustrated in FIG. 1.

It comprises means for three-dimensionally treating 100 a zone to be treated of the body of a subject, said means 100 comprising means 200 for forming a volume of adipose tissue to be treated, means 300 for generating ultrasound and means 400 for generating electric pulses placed on the volume of adipose tissue thus formed.

The means 300 for generating the ultrasound and the means 400 for generating the electric pulses are placed and distributed on a supple membrane 500 having certain elasticity.

It is designed to cover the interior of the means 200 for forming the volume of adipose tissue for applying the means for generating the ultrasound and the electric pulses 300 and 400 to the skin of the subject and, consequently, to the volume of adipose tissue formed, as it will be described hereinbelow in relation to FIGS. 2 and 3.

More precisely, on this supple membrane 500 are placed cutaneous electrodes 410 designed to guide electric pulses I in the volume in question and cutaneous transducers 310 designed to pass ultrasound waves U through the volume in question, for example ultrasound waves, typically theracanic ultrasound waves (ultrasound frequencies used in medicine).

The use of ultrasound U and electric pulses I can be done locally on the volume of adipose tissue formed simultaneously, successively or alternately for treating cellulite and fatty masses.

Ultrasound U transmits vibrations to the fatty cells of the fatty layer, which activate the phenomenon of dissolution of fat from these cells, as well as lipidic exocytosis of the latter, that is, rejection of fats from the cells.

The possibility of associating an ultrasound U and electric pulse I application on the same volume of adipose tissue improves these results.

On the one hand, sending sequences of electric pulses I improves the efficacy of the action of low-frequency ultrasound U on lipidic exocytosis of fatty cells.

In fact, sending electric pulses I evacuates water which would stagnate in the volume of treated adipose tissue. The treatment of low-frequency ultrasound U which would have had a dispersed action on the lipidic medium and the aqueous medium present can then be concentrated on the lipidic medium and be more efficacious.

On the other hand, the use of sequences of specific electric pulses I also boosts the blood flow of the venous system and locally activates lymphatic circulation which stimulates lymphatic drainage, thus reducing excessive infiltration of water and evacuating lipidic rejection of cells to the liver, which then eliminates them.

At the level of the volume of treated adipose tissue, the association of ultrasound U sequences and electric stimulation of muscles causes a series of instances of exocytosis of lipids of fatty cells and drainage, prompting evacuation of lipidic discharge throughout treatment.

This effectively limits any local or even temporary accumulation of lipids which might risk being reabsorbed by the fatty cells of the fatty layer.

The results obtained on treating cellulite and fatty masses of the subject are strongly improved and durable.

Means for Forming a Volume of Adipose Tissue

Treating cellulite and fatty masses three-dimensionally performed by means of forming volume of adipose tissue 200 offers the possibility of using several sources of ultrasound in phase 310 guiding ultrasound waves converging on the same target zone of adipose tissue.

To the extent where the generator delivers signals in phase or with a slight difference in phase to these ultrasound sources 310, the effects of ultrasound waves U are added to the zone in question, generating greater energy focused on the volume of adipose tissue treated.

While keeping the intensity below regulatory limits of ultrasound emission intensity, the energy generated at the level of the volume of adipose tissue treated is much more significant at equal intensity, making treatment more efficacious.

The sum of ultrasound waves U favours exocytosis of lipids within the volume of adipose tissue.

In order to optimise the effects of ultrasound waves U generated in phase, it is possible to have the transducers 310 adapted to the means of forming the volume of adipose tissue to be treated 200.

In this way, the transducers 310 can be oriented in such a way that the ultrasound waves converge on the centre of the volume of adipose tissue formed.

Concerning more precisely the means of forming the volume of adipose tissue 200, they can comprise aspiration means 210 (suction) and/or mechanical pincer means 220 of adipose tissue, such as illustrated respectively in FIGS. 2 and 3.

By way of advantage, these continuous or sequential suction means 210 or mechanical pincer means 220 contribute via effect mechanical to expelling lipids from adipocytes, activate blood and lymphatic circulation and reduce fibrosis of the connecting tissue surrounding the lobes of adipose tissue.

They will apply drop in pressure or external mechanical pressure to the volume of adipose tissue which they mobilise.

This pressure or drop in pressure participates in a fragilising action of the membranes of fatty cells and favours their bursting and rejection of lipids from the fatty cells.

This makes it possible to accelerate and improve lipidic exocytosis of fatty cells created by treatment combined with low-frequency ultrasound U and electric pulses I. The suction means 210 or mechanical pincer means 220 formed by mobile rollers, which can be motorised, also improve drainage lymphatic of discharge from lipids.

As illustrated in FIG. 2, the suction means 210 are in the form of an assembly comprising a casing 211 fed by a vacuum pump 212 to create localised drop in pressure necessary for suctioning adipose tissue.

Throughout this drop in pressure, blood vessels dilate and transport fats via the blood to evacuate them.

Depression, adjustable by the user, is preferably comprised between 100 and 600 millibars, typically between 150 and 300 millibars.

The casing 211 has an internal chamber 213 open to its face and designed to be in contact with the skin of the subject.

This chamber 213 extends transversally over the entire length of the casing 211.

It can be in semi-cylindrical form or even demi-spherical, having the shape of a bell.

It preferably has an internal diameter of less than 15 cm, typically less than 10 cm.

However, others variant embodiments concerning the form or dimensions of the chamber 213 can be provided.

The supple membrane 500 comprising the transducers 310/electrodes 410 assembly is designed to coat the concavity of the chamber 213 to be in contact with the skin of the subject.

To boost the effects of ultrasound U generated in phase, the transducers 310 of the supple membrane 500 are oriented in such a way that the ultrasound waves U converge on the axis central of the semi-cylinder or the centre of the demi-sphere of the volume of adipose tissue formed after suction or toeing-in.

In addition, any transducer 310 of the supple membrane 500 directed perpendicularly to the non-treated surface of the skin of the subject can be withdrawn to avoid sending ultrasound waves U to its internal organs. The other transducers 300 are sufficiently numerous for generating considerable energy within the treated volume.

In addition, a variant embodiment also provides means of automatic displacement of the suction means 210 or pincer means 220.

In this way, rollers controlled by appropriate means can be placed on the external periphery of the chamber 213 in contact with the skin of the subject. These rollers displace the suction means 210 at predefined time intervals to change volume of adipose tissue to be treated.

As illustrated in FIG. 3, the mechanical pincer means 220 as such comprise a supple plate 221 of rectangular shape delimited by two rollers 222 and 223 on these opposite longitudinal ends.

The mechanical pincer means 220 allow the user to delimit a volume of adipose tissue to be treated by a fold of skin by mechanical pincer means of the plate 221 by means of the rollers 222 and 223.

The supple membrane 500 comprising the transducers 310/electrodes 410 assembly is designed to coat the face of the plate 221 placed in contact with the skin of the subject.

In a variant embodiment of the device 10, the suction means 210 and the mechanical pincer means 220 can be used in alternation to create the volume of adipose tissue to be treated.

According to another embodiment, an exploded view of which is illustrated in FIG. 4, the means 200 for forming a volume of adipose tissue to be treated comprise a chamber 213 having a hemispherical form or the form of a bell, an intermediate element 215 and a base 218, these elements being interconnected by clipping or by screwing.

The intermediate element 215 and the base 218 each comprise a supple membrane, respectively referenced 502 and 504 in FIG. 4, and supports surrounding these membranes 502 and 504, respectively intermediate support 216 and base support 219. The intermediate element 215 further comprises a locking element 217 allowing affixing of the intermediate membrane 504 during assembly of the means 200. The supple membrane 504 is kept in position between the intermediate support 216 and the base support 219.

In this embodiment, the two supple membranes 502 and 504 have distinct roles;
the supple membrane 504 of the base 218 is for one-off use; it is replaced after each subject is treated; it is this supple membrane 504 which is in contact with the zone to be treated. The fineness of the membrane is advantageous relative to elasticity, to the extent where it is for one-off use; it is typically made of polyurethane or any other material having equivalent properties.
the supple intermediate membrane 502 serves as support to the transducers which are placed on this supple intermediate membrane 502. This supple intermediate membrane 502 is not directly in contact with the zone to be treated, due to the presence of the supple membrane 504 of the base 218; the allergising character of this material is therefore not troubling. This supple intermediate membrane 502 is made of latex, for example.

Typically, the electrodes 410 are placed on or at the periphery of the face of the base 218 in contact with the zone to be treated. The transducers 310 as such are placed on the supple intermediate membrane 502, on the face of this membrane 502 opposite the zone to be treated.

Several variants are possible; the electrodes 410 can also be placed on the same supple membrane 502 as the transducers 310, or on a supplementary supple membrane distinct from that serving as support for the transducers 310, not illustrated in the figures.

Contrary to known means which utilise a rigid support to form the volume of adipose tissues to be treated, use of a supple membrane 500 ensures that the transducers 310 are in contact with the epidermis at all times, and that this is before suction, during suction and after suction.

The fact of having a contact between the transducers 310 and the epidermis prior to suction allows:
analysis of the thickness and the texture of the fatty tissue to be treated (imagery effect, or sonar, echographic type), or the active or reactive energy consumed to adapt, automatically or not, the parameters (intensity of ultrasound, time of activity relative to rest time, treatment time of the suctioned zone, number of successive passes to be made on the suctioned zone . . . )
adaptation of the suction to all skins, irrespective of their elasticity on the one hand, and to all thicknesses of fat on the other hand. For example, in case of lack of elasticity and/or minimal fat thickness, suction is limited to prevent pain, while providing efficacious treatment.

Known means which utilise a rigid support to form the volume of adipose tissue to be treated exhibit several disadvantages relative to this solution, especially the fact that the tissues must be suctioned until they are in contact with the transducers. Yet, experimenting has shown that:
the suctioned skin mainly blocks the source of the suction, which can prevent placement of the rest of the tissues in contact with the transducers 310 fixed to the internal face of the bell.
when tissues lack elasticity, the suction necessary for the tissues to completely fill the interior of the bell causes pain. In the embodiment employing a supple membrane, it is not indispensable to completely fill the bell since the transducers 310 are initially in contact with the epidermis, and it is therefore possible to conduct treatment by decreasing suction (the transducers are then in an oblique position, but they converge all the same, allowing efficacious treatment),

However, when the transducers 310 are on the internal face of the bell, and if the bell is not suctioned to the point where it is completely full, the tissues do not make contact with the transducers 310, therefore the treatment cannot be completed correctly. And if a shallower bell is used, when the adipose tissues are thick, treatment occurs solely on the upper part of the adipose tissue, therefore only on a superficial strip of fat and not the whole thickness of the fatty tissue. Also, the volume treated is reduced, requiring treatment time to be prolonged or treatment sessions multiplied.

Also, the height of the bell determines the size of the transducers 310, therefore a shorter bell must have smaller transducers 310, and thus reduces the volume treated as far as possible.

It is also noted that conventional devices comprise an opaque bell, not ensuring that the epidermis is in contact with the transducers 310, whereas in the embodiment proposed the epidermis is in contact with the transducers 310 via the support membranes as soon as the device 10 is set on the body of the subject to be treated. In the present device, the bell of plastic material and/or the membranes can be made of translucid material, thus ensuring that the epidermis is in contact with the transducers 310.

In addition, the fact of having contact with the epidermis prior to suction visualises the placement of the transducers 310 on the epidermis, and consequently avoids sensitive zones such as scars, and ensures optimal and reproducible penetration of the ultrasound through the tissues.

According to a particular embodiment, the volume of the bell is over 260 cubic centimetres, and the volume treated at each apposition is over 157 cubic centimetres.

To have the bell ensure several functions, the invention comprises a bell in two parts: an internal bell ensuring suction and another covering it and comprising the electronics and terminals.

The electronics and terminals can also be located in a casing comprising the feed of the device, pump, etc.

The internal bell is hollow on its internal face with multiple furrows which converge on the suction orifice so that air can be suctioned evenly for the entire volume without the membrane risking blocking the suction orifice, thus optimising treatment conditions by placing the transducers 310 opposite in pairs.

In addition, the means for forming the volume of adipose tissue 200 also boost the effect of ultrasound U on the fatty cells by using, as already mentioned, an emission power of ultrasound below the limit tolerated on the body of 3 Watts/cm2.

They also use transducers 310 smaller in size by conserving good treatment efficacy, thus reducing production costs of the device 10.

Transducers/Electrodes Assembly

As mentioned earlier, an assembly of transducers 310 and electrodes 410 is placed on the means 200 forming the volume of adipose tissue to be treated due to the supple membrane 500 to be applied on or at the periphery of the volume of adipose tissue in question.

This assembly preferably comprises at least two electrodes 410 and two transducers 310, such as illustrated in FIGS. 2, 3 and 4.

The electrodes 410 guiding the trains of electric pulses I and the transducers 310 guiding the ultrasound U are alternated on the supple membrane 500 forming diverse patterns.

In this way, as illustrated in FIG. 2, the electrodes 410 and the transducers 310 can be placed so as to form juxtaposed concentric circles. For example, the electrodes 410 surround the transducers 310 or inversely.

In accordance with another embodiment illustrated in FIG. 3, they are placed to form a beehive pattern.

According to another embodiment, the supple membrane 500 can form the electrodes 410 in which through holes allow passage of the transducers 310.

According to another embodiment illustrated in FIG. 4, the electrodes 410 are placed on the periphery of the base 218, whereas the transducers 310 are placed on the supple intermediate membrane 502, or more generally, on the supple membrane 500 when the device comprises only one supple membrane 500.

Ultrasound and electric pulses are generated on the volume of adipose tissue formed, or around the latter when the means for generating electric pulses are located on a support located at the periphery of the adipose tissue formed.

The transducers 310 are preferably placed on the supple membrane 500 so as to be equidistant from one another to optimise distribution of the ultrasound waves U in the tissues treated.

In addition, such as illustrated in FIGS. 1 to 3, the assembly of transducers 310 and electrodes 410 is connected to one or more generators 600 for supplying electric pulses I and feeding the transducers supplying the ultrasound U, as well as one or more amplifiers. The assembly comprising the generator or the generators and the amplifier or the amplifiers constitutes an electronic module for managing the ultrasound and electrostimulation.

This generator 600 supplies electric pulses I via trains with an electric current defined as a pulsed alternative current.

The electric current applied through the electrodes 410 has an intensity of between 5 μA and 100 mA, typically between 100 μA and 70 mA.

The generator 600 can also allow different ultrasound sources such as the transducers 310 to send ultrasound waves in phase. Alternatively, it can be provided that this function is ensured by another generator (not illustrated).

To avoid heating the skin of the subject, the ultrasound U are sent via pulse trains.

The ultrasound emission power is as such typically defined to be less than 3 Watts/cm2 in compliance with current legislation to avoid cutaneous lesions of the subject.

It should be noted that the electrodes 410 and/or the transducers 310 can be for one-off use, replaced at each treatment for hygienic reasons.

According to a preferred embodiment, gel ensures perfect contact with the epidermis of the body of the subject treated; a cream can also be used.

The supple membrane 500 can be covered by gel and/or adhesive and can be for one-off use.

The transducers 310 can be made of ceramic material, or PVF film (polyvinyl fluoride), this material making, when suction is operating, a continuous crown which completely surrounds the zone to be treated.

Other Means of the Device

In addition, the device 10 can comprise various sensors or control devices, such as illustrated in FIG. 1.

In this way, it can comprise cooling means 610 of the volume of adipose tissue treated in contact with the assembly transducers 310/electrodes 410 so as to generate vasoconstriction and compensate local heating due to ultrasound U to which this volume is subjected.

In addition, it can also comprise heating means 620 of the volume of adipose tissue treated.

In this way, it is possible to alternatively heat and cool the volume of adipose tissue treated in order to activate blood circulation.

This thermal stimulation also activates the chemical hydrolysis transformation cycle of triglycerides into fatty acids used in the pyruvic acid cycle or they are transformed into pyruvates and consumed by muscle fibres.

Also, thermal stimulation alternating heat and cooling also releases neuromediators such as adrenalin and noradrenalin which are captured by adipocyte receptors, especially beta receptors, and stimulate them to trigger lipolysis.

The device 10 can also comprise temperature-detection means 630 at the level of the volume of adipose tissue treated, adapted to cut the generator or the generators 600 in the event of excessive temperature on the skin of the subject.

In addition, the device can contain sensors which give information on the thickness and density of the volume of tissue to be treated, or the active or reactive energy consumed, and if necessary adjust the parameters of the device. This regulation can also be done using information on the energy, active and reactive, received by the transducers.

The device 10 can also comprise a processor 640 for controlling the assembly of means employed, as well as display and safety/protection means 650 of the patient.

During treatment these display means 650 could especially display the duration of treatment selected and/or remaining as well as the characteristics of ultrasound and electric pulses, such as depth of penetration, frequency and/or current, power of drop in pressure. Non-limiting examples are use of a liquid-crystal monitor.

According to a particular embodiment, acoustic lenses are added in front of each transducer 310 to modify the diffusion angle of ultrasound to optimise its convergence. In this way, when the tissues are not very elastic, causing limited suction and therefore the fact that the transducers 310 are not exactly opposite, acoustic lenses level out the ultrasound beams to make them converge.

The device 10 can also comprise an integrated contact gel dispenser for ultrasound.

In fact, as mentioned previously, application of gel improves transmission of ultrasound U to the tissues of the subject. The gel can be neutral, or may even contain active ingredients whereof penetration through the skin to the fatty tissues will be widely boosted by ultrasound.

The bell is formed from two walls between which a container of this gel can be placed.

The base of the bell is formed by the support of the internal membrane (supporting the transducers), itself in contact with the support of the external membrane (supporting the electrodes).

The gel container is connected to the support of the external membrane via the support of the internal membrane. On its lower face (in contact with the subject) the support of the external membrane comprises multiple small orifices located on its internal periphery.

In response to pressure from a piston placed on the bell, or the envelope of the bell itself if the latter is made of supple material, such as silicon for example, the gel is expelled on the lower face of the external membrane to optimise the interface with the epidermis of the subject.

The pressure can be manual, or generated automatically prior to each suction event.

According to another configuration, the membrane can be pre-coated, on the face in contact with the skin, by contact gel, with or without active ingredient.

The device can also comprise a guide system.

In fact, to treat a complete zone, the bell has to be applied a number of times on the same place at intervals of several minutes.

Also, the bell has to be positioned with partial covering to compensate the adipose parts located under the transducer once suction is completed, therefore not insonified.

The operator delimits the zone to be treated by tracing marks (crosses) on the skin of the patient, and a camera placed above the patient identifies the limits of the field.

The informatics system measures the size of the zone to be treated using the size of the bell as reference (calibration), therefore irrespective of the distance between the camera and the patient, then determines the successive placements of the bell and the total treatment time.

The operator places the bell on the site indicated on the monitor in overprint of the operating field. After each application, the software indicates the new placement of the bell, the placement having been treated appearing increasingly opaque on the monitor during successive passes.

If the patient wants to move, the system can be interrupted, then automatically recalculated and resumed on return of the patient.

Other guide systems are feasible; examples are an LED matrix, or even motorised laser beam placed above the zone to be treated and illuminating the placement where the operator must place the bell, or any other adapted means.

The guide system can also mark the treated zone relative to anatomical marks (for example, skin folds, navel, moles etc.) and save them in the patient file to be able to indicate them to the operator during subsequent treatment of a relevant zone or of the same zone, if necessary.

### 2. Treatment Process

A process for executing the device 10 for treating cellulite and fatty masses and especially for cosmetic treatment will now be described. FIG. 5 illustrates these different steps.

Initially, in a first optional step 20, a gel is applied to the skin of the subject treated to improve transmission of ultrasound U to the tissues of the subject.

In a second step 30, a volume of adipose tissue to be treated is formed.

For this, either the suction means 210 of the mechanical pincer means 220 such as described previously are used.

It should be noted that suction with adjustable power must be limited over time to prevent the dermis from lifting off the skin, as after treatment this would cause the appearance of ecchymoses such as reddish suction marks.

Suction lasts preferably less than two minutes on the same zone of adipose tissue treated.

In an embodiment of the process, the supple membrane 500 supporting the transducers 310 and the electrodes 410 is previously deposited flat on the skin. The supple membrane 500 is kept on the skin by attachment means, such as adhesive, for example.

The suction means 210 or the mechanical pincer means 220 are positioned above the supple membrane 500.

In this way, during suction, the supple membrane 500 conforms to the volume formed. In other words, the supple membrane 500 covers the semi-cylindrical or semi-spherical volume formed. The supple membrane 500 keeps the transducers 310 and the electrodes 410 (if placed on the supple membrane 500) in contact with the skin during suction.

When treatment is performed on a subject with limited tissue elasticity, depending especially on the thickness of the layer of fat of the subject, suction of tissues to form the semi-cylindrical or semi-spherical volume risks being painful and the dermis can deform.

Thanks to the supple membrane 500, the drop in pressure can be reduced to limit the tension on the tissues while keeping the electrodes 410 and the transducers 310 properly applied to the volume of adipose tissue to be treated.

The membrane thus allows pain-free treatment which can be performed on all subjects, irrespective of the elasticity of their skin.

The membrane-transducers-electrodes assembly can be supported by a rigid structure, for example plastic or metal, which can be fixed for example by screwing or clipping onto the bell or the suction cylinder. For greater ease of use, the various wires of the transducers and electrodes can be combined into a single strand to be connected to its counterpart coming from the electricity generator. The membrane-transducers-electrodes-support assembly can be identified, for example by a RFID chip, a barcode or other means, so that its use is recognised by the system. In this way, for hygienic reasons, it is possible to authorise treatment only with a membrane-transducers-electrodes assembly never having previously been used, or else having been previously used only to treat the very patient who is going to receive more treatment. In this case, recognising the membrane-transducers-electrodes assembly automatically displays the patient file on the information console.

If there is no suction, or if toeing-in relays suction, the membrane can be put in place before or after toeing-in.

In another embodiment, it can be provided that the supple membrane 500 coats the interior of the means 200 for forming the volume. In this case, the supple membrane 500 is either permeable in that it lets the suctioned air pass through, or it comprises one or more orifices adapted to allow passage of air to suction nozzles of the means 200.

The electrodes 410 and the transducers 310 applied to the skin of the subject will then guide respectively sequences of electric pulses I and ultrasound U predetermined on the volume of adipose tissue formed.

In this way, in a step 40, the transducers guide ultrasound U generated at a frequency of between 20 kHz and 3 Mhz and at an intensity of between 0.5 and 3 watts/cm² on the volume of adipose tissue.

It should be noted that the frequency and intensity of this ultrasound U can change throughout treatment either manually by choice of the operator, or automatically as a function of the information on thickness, density and temperature recorded on the treated zone, or on consumption of active or reactive energy of the transducers 31.

To improve and accelerate exocytosis of lipids of adipose cells, electric stimulation is carried out at the same time as sending of the ultrasound sequences (step 50).

However, it can also be done successively or alternately.

A current is then applied through the electrodes 410 at an intensity of between 5 μA and 100 mA, typically between 100 μA and 70 mA on the volume of tissue to be treated.

The electric pulses I generated then stimulate the smooth muscles, by the volume of adipose tissue treated.

Alternating the contraction and relaxing of the smooth muscles increases the blood flow of the venous system and locally stimulates lymphatic circulation evacuating this lipid discharge from cells to be eliminated.

Time sequences of electric pulses I are preferred to continuous application of electric pulses I.

In a final step 60, due to either automatic or manual displacement means, the three-dimensional treatment means 100 are then displaced to another zone of the body of the subject to be treated.

Supplementary steps are possible to optimise the process, these steps intervening before and/or after suction.

Before treatment, the bell is placed, the transducers being on the supple membrane 500 flat on the skin, without suction. Analysis of tissue is performed by ultrasound, such as echography: one or more transducers sends ultrasound whereof the return (echo) is modified as a function of the elements encountered, and is recorded to indicate thickness and density of the tissues penetrated (dermis, adipose and fibrous tissues, distance from subjacent muscle etc.), or the active or reactive energy consumed. This information will be utilised to automatically adjust the different treatment parameters (level of drop in pressure, frequency and intensity of ultrasound, duration of insonification . . . ).

This analysis is possible only if the transducers are in direct or indirect contact with the epidermis (for example placed flat on the epidermis), therefore because of the supple membrane 500 support of the transducers 310.

Analysis can also determine the dimension of adipocytes of the tissue to be treated (which can vary by 115% between 70μ and 150μ) such that equipment generates the ultrasound frequency corresponding to the resonating of these cells to optimise the phenomenon of cavitation and the efficacy of ultrasound on these cells. It is possible for example to have four transducers 310 at a frequency determined as a function of the thickness of the adipose tissue, and four other transducers 310 at a frequency determined as a function of the size of the adipocytes.

It is also possible to precisely measure the thickness and density of the adipose tissue or the active or reactive energy consumed once suction is completed to adapt the intensity of ultrasound to be delivered.

This adjustment can be made either by modifying the power of the amplifier, or by modifying the frequency, the result of which is generating a different efficacious intensity.

In addition, each transducer 310 has an original frequency, for example 1 MHz, which corresponds to the maximum yield put out when it is excited at this frequency. But, this optimal frequency varies as a function of the elements in contact with the transducer 310: welded electric wires, supple membrane 500, tissues to be treated, and according to the presence, correct or not, of contact gel.

Due to a diagnostics autotest, the frequency can be modified to rediscover the optimal yield. The excitation frequency of the transducers 310 can thus be modified to obtain a more or less substantial intensity as a function of the analysis of tissues of the patient.

This can be done prior to treatment due to the position of the transducers 310 flat on the skin prior to suction, or by measuring the active and reactive energy consumed.

Similarly, the equipment can generate the optimal frequency so that the ultrasound causes a cavitation phenomenon of the adipocytes as a function of their size (F=Pi/size, where “F” is the optimal frequency for the ultrasound to cause a cavitation phenomenon of the adipocytes, and “size” is the size of the adipocytes).

The amplitude of the drop in pressure to be provided to reach the cavitation threshold depends on several parameters: frequency, viscosity of the medium, presence of microparticles or dissolved gases:
the higher the frequency of the sound, the shorter the period of drop in pressure, and it can be too short to form a cavity;
the higher the viscosity of the medium (therefore internal cohesion of the liquid), the more difficult the cavitation to be produced due to the fact that the particles are more difficult to separate;
the presence of microparticles or dissolved gases are just as many elements of heterogeneity which constitute disruption zones of the liquid medium favouring formation of cavitation bubbles, specifically lowering of the cavitation threshold (or Blake threshold), that is, of the minimal power from which cavitation takes place.

The electronic management module for ultrasound and electrostimulation (generator-amplifier etc.) can either be placed in the casing of the equipment, for example with the suction elements to form a volume of adipose tissue and calculation means allowing inter alia conducting diagnostics, or, due to optimisation of ultrasonic output by autotest, be sufficiently miniaturised to be placed in the bell itself.

Therefore, the “antenna” effect of two metres around the terminals (wires, cables, . . . ) which connect the equipment to the bell is avoided, and the risk of electromagnetic perturbation which could destabilise other instruments is reduced, making it easier to have equipment comply with Electromagnetic Accounting standards as required for medical instruments.

1. A device (10) for treating cellulite and fatty masses comprising means (200) for forming a volume of adipose tissue on a zone to be treated of the body of a subject, means (300) for generating ultrasound and means (400) for generating electric pulses on the volume of adipose tissue formed or at the periphery of the volume of adipose tissue formed, said device (10) being characterised in that the means for generating ultrasound (300) are supported by a supple membrane (500) connected to the means (200) for forming a volume of adipose tissue, and adapted so that when the device (10) is placed on the zone to be treated of the body of a subject, the means (300) for generating ultrasound remain in contact with the volume of adipose tissue to be treated, and in that the means for generating electric pulses (400) are placed on or at the periphery of a face of the device (10) in contact with the zone to be treated

2. A device (10) for treating cellulite and fatty masses comprising means (200) for forming a volume of adipose tissue on a zone to be treated of the body of a subject, means (300) for generating ultrasound and means (400) for generating electric pulses on the volume of adipose tissue formed, said device (10) being characterised in that the means for generating ultrasound (300) and the means for generating electric pulses (400) are supported by a supple membrane (500) connected to the means (200) for forming a volume of adipose tissue, and adapted so that when the device (10) is placed on the zone to be treated of the body of a subject, the means (300) for generating ultrasound and the means (400) for generating electric pulses remain in contact with the volume of adipose tissue to be treated.

3. The device as claimed in any one of the preceding claims, characterised in that the means for generating electric pulses (400) comprise at least two cutaneous electrodes (410) designed to guide electric pulses in the volume of adipose tissue and the means for generating ultrasound (300) comprise at least two cutaneous transducers (310) designed to guide ultrasound in the volume of adipose tissue.

4. The device as claimed in claim 3, characterised in that the electrodes (410) and the transducers (310) are placed on the supple membrane (500) so as to form a pattern in which the electrodes (410) surround the transducers (310) or inversely.

5. The device as claimed in any one of claims 1 or 2, characterised in that the means for forming a volume of adipose tissue (200) comprise suction means of adjustable power (210) for adipose tissue.

6. The device as claimed in any one of claims 1 or 2, characterised in that it further comprises cooling means (610) and/or heating means (620) of the volume of formed adipose tissue to be treated.

7. The device as claimed in any one of claims 1 or 2, characterised in that it further comprises temperature-detection means (630) at the level of the volume of formed adipose tissue to be treated.

8. The device as claimed in any one of claims 1 or 2, characterised in that it further comprises means for detecting thickness and density (630) at the level of the volume of formed adipose tissue to be treated allowing adjustment of the parameters of ultrasound and current.

9. The device as claimed in any one of claims 1 or 2, characterised in that the means (200) for forming a volume of adipose tissue on a zone to be treated of the body of a subject comprise two membranes, one being adapted to be directly in contact with the zone to be treated of the body of the subject and to be replaced after each subject to be treated, and the other being adapted to serve as support to the transducers (310) and not be in direct contact with the zone to be treated of the body of a subject.

10. The device as claimed in any one of claims 1 or 2, characterised in that it comprises a contact gel dispenser for ultrasound integrated with the means (200) to form a volume of adipose tissue.

11. The device as claimed in any one of claims 1 or 2, characterised in that it further comprises means adapted to execute echography of the zone to be treated, and measure the active or reactive energy consumed to regulate treatment parameters, such as:
level of drop in pressure,
frequency of ultrasound,
intensity of ultrasound,
duration of insonification,
rest time of ultrasound relative to work time,
intensity of electro-stimulation pulses delivered,
as a function of the elements identified by echography, such as thickness and density of tissues penetrated.

12. The device as claimed in any one of claims 1 or 2, characterised in that it further comprises a guide system adapted, at each application of the device on the zone to be treated of the subject, to identify the position of the device, and the number of applications of the device made on this position during treatment.

13. The device as claimed in any one of claims 1 or 2, characterised in that it further comprises means adapted to identify the frequency of transducers allowing optimal yield, said identification being made when the transducers are placed flat on the zone to be treated.

14. The device as claimed in any one of claims 1 or 2, characterised in that the bell of plastic material and/or the membranes are made of translucid materials adapted so that the position of the transducers on the zone to be treated of the subject is visible when said device is placed on the zone to be treated of the subject.

15. A process for treating cellulite and fatty masses, characterised in that it comprises a step (30) for forming a volume of adipose tissue on a zone to be treated of the body of a subject followed by a step of placing a supple membrane (500) connected to a support in contact with the volume of adipose tissue (200) thus formed, said supple membrane (500) supporting means for generating ultrasound (300), and said supple membrane (500) or said support supporting means for generating electric pulses (400) and followed by a step (40, 50) for generating ultrasound and for generating electric pulses on the volume of adipose tissue formed or around the latter when the means for generating electric pulses are located on a support located at the periphery of the adipose tissue formed.

16. The process as claimed in claim 15, characterised in that it comprises an additional step for analysis of tissues to be treated, said analysis being performed by the emission of ultrasound whereof the return is modified as a function of the elements encountered, and is recorded so as to indicate the thickness and density of the tissues penetrated.

17. The process as claimed in claim 16, characterised in that it comprises an additional step of automatic adjustment as a function of the active or reactive energy consumed or other information on tissues obtained by echography of the different treatment parameters, including:
level of drop in pressure,
frequency and intensity of ultrasound,
duration of insonification,
intensity of electro-stimulation,

18. The process as claimed in any one of claims 16 or 17, characterised in that said analysis comprises a step for measuring the dimension of adipocytes of the tissue to be treated, as well as a step for regulating the emission frequency of ultrasound so that their emission frequency corresponds to the resonance frequency of these adipocytes.

19. The process as claimed in claim 15, characterised in that the generation of ultrasound and the generation of electric pulses are carried out simultaneously.

20. The process as claimed in claim 15, characterised in that it further comprises a step of application of contact gel for ultrasound on the volume of tissue to be treated.
